(19) United States
(12) Patent Application Publication
(10) Pub. No.: US 2014/0259224 A1
(43) Pub. Date: Sep. 11, 2014

(54) TRANSGENIC PLANTS WITH ENHANCED TRAITS AND METHODS OF PRODUCING THEREOF

(71) Applicants: Nicole Christiansen, Berlin (DE); Stefan Henkes, Potsdam (DE); Ganesh Kumar, Chesterfield, MO (US); Amanda McClerren, St. Charles, MO (US); Robert Meister, St. Peters, MO (US); Marie Petracek, Glendale, MO (US); Sasha Preuss, St. Louis, MO (US); Richard Trethewey, Research Triangle Park, NC (US); Qingzhang Xu, Johnston, IA (US)

(72) Inventors: Nicole Christiansen, Berlin (DE); Stefan Henkes, Potsdam (DE); Ganesh Kumar, Chesterfield, MO (US); Amanda McClerren, St. Charles, MO (US); Robert Meister, St. Peters, MO (US); Marie Petracek, Glendale, MO (US); Sasha Preuss, St. Louis, MO (US); Richard Trethewey, Research Triangle Park, NC (US); Qingzhang Xu, Johnston, IA (US)

(73) Assignees: BASF PLANT SCIENCE COMPANY GMBH, Ludwigshafen (DE); MONSANTO TECHNOLOGY L.L.C., St. Louis, MO (US)

(21) Appl. No.: 14/006,618

(22) PCT Filed: Mar. 21, 2012
(86) PCT No.: PCT/US12/29885

§ 371 Date: Jan. 3, 2014

Related U.S. Application Data
(60) Provisional application No. 61/467,766, filed on Mar. 25, 2011.

(51) Int. Cl. C12N 15/82

(52) U.S. Cl. CPC C12N 15/8241; C12N 15/8245; C12N 15/8246
USPC 800/284; 800/278; 435/419; 800/312; 800/314; 800/306; 800/320; 800/320.1; 800/320.2; 800/320.3; 800/322; 800/298; 530/378; 530/376; 530/370; 554/9; 530/375; 530/372; 530/377; 426/615; 426/635; 426/636; 426/630; 426/634; 426/622; 426/623

(57) ABSTRACT

The present disclosure provides methods of producing a plant that exhibits an enhanced trait selected from the group consisting of altered hexose sugar level, altered starch level, altered sucrose phosphate synthase activity, altered ureide level and delayed senescence, as compared to a control plant. The present disclosure also provides transgenic cells, plants, plant parts, seeds, progeny plants, products or commodity products produced by this method.

### CROSS REFERENCE TO RELATED APPLICATIONS

This application claims the benefit of U.S. Provisional Application No. 61/467,766, filed on Mar. 25, 2011, incorporated herein by reference in its entirety.

### INCORPORATION OF SEQUENCE LISTING

The sequence listing that is contained in the file named “57775B_ST25.txt”, which is 15 kilobytes as measured in Microsoft Windows operating system and was created on 19 Mar. 2012, is filed electronically herewith and incorporated herein by reference.

### FIELD OF THE INVENTION

The present disclosure relates to the field of plant molecular biology and plant genetic engineering. Specifically, the present disclosure provides methods of producing a plant that exhibits an enhanced trait. Also disclosed are transgenic cells, plants, plant parts, seeds, progeny plants, plant products, or commodity products produced by the methods.

### BACKGROUND OF THE INVENTION

World demand for grains, driven by increased population, higher global per-capita incomes, and increased demand for protein, is predicted to increase by seventy percent (Rosegrant and Cline, 2003) (FAO, 2009) by the year 2050. While the overall agricultural productivity has increased in the preceding decades due to advances in breeding and new agricultural practices including seed treatment, there is still a need for new technologies to meet the challenge of increased demand for grains.

Commercially valuable crop plants in the natural environment often grow under suboptimal or unfavorable conditions, such as at extreme temperatures, variable water availability, or with a limited supply of soil nutrients, which may significantly affect a plant's yield. While little can be done to change the natural environment under which the crop plants are grown, a plant's traits, including its biochemical, developmental, or phenotypic characteristics that enhance yield or tolerance to various abiotic stresses, may be controlled through a number of cellular processes. One important way to manipulate that control is through proteins that influence the expression of a particular gene or sets of genes. The Arabidopsis BBX32 (AtBBX32) protein of the present disclosure modulates plant diurnal processes, such as source capacity regulation and utilization of photosynthesis products, which improves capacity for reproductive development, resulting in higher yield (U.S. Pat. No. 7,692,067).

### SUMMARY OF THE INVENTION

The present disclosure provides methods for producing a plant that exhibits an enhanced trait selected from the group consisting of altered hexose sugar level, altered starch level, altered sucrose phosphate synthase (SPS) activity, altered ureide level and delayed senescence, as compared to a control plant. More specifically, the method comprises 1) providing a recombinant nucleic acid encoding a polypeptide, wherein the polypeptide comprises a conserved domain having the amino acid sequence of SEQ ID NO: 7; 2) introducing into a plant cell the recombinant nucleic acid to produce a transgenic plant; and 3) growing the plant with an enhanced trait. Transgenic cells, plants, plant parts, seeds, progeny plants, products or commodity products produced by this method are aspects of the disclosure, and may be from any plant species, or may be from any crop plant, or may be selected from the group consisting of soybean, cotton, canola, alfalfa, corn, rice, wheat, sunflower, barley, millet, sorghum, sugar beet, sugarcane and vegetables.

The present disclosure further provides a commodity product from the transgenic plant of the disclosure, including, but being not limited to, whole or processed seed, animal feed, oil, meal, mill, flour, flake, bran, protein concentrate, soy protein isolates, hydrolyzed soy protein, biomass and fuel products.

The foregoing and other aspects of the disclosure will become more apparent from the following detailed description.

### BRIEF DESCRIPTION OF THE DRAWINGS

FIG. 1. Alignment of protein sequences and identification of domains and motifs. Solid line box represents the B-box domain, whereas dashed line boxes represent common motifs among the three genes.

FIG. 2. Comparison of leaf senescence between AtBBX32 transgenic plants and their negative segregant or wild type control plants. Leaf senescence was visually scored during the late reproductive stage of development, with 1 representing no senescence, 6 representing full senescence. Error bars represent standard error; “*” illustrates statistical difference from the wild type control at p≦0.05; “†” shows statistical difference from the negative segregant control at p≦0.05.

### BRIEF DESCRIPTION OF THE SEQUENCES

SEQ ID NO: 1 provides the coding sequence of AtBBX32 from Arabidopsis thaliana (AtBBX32).
SEQ ID NO: 2 provides the coding sequence of BBX52, an AtBBX32 homolog from Glycine max (GmBBX52).
SEQ ID NO: 3 provides the coding sequence of BBX53, another AtBBX32 homolog from Glycine max (GmBBX53).
SEQ ID NO: 4 provides the amino acid sequence of AtBBX32 protein.
SEQ ID NO: 5 provides the amino acid sequence of GmBBX52 protein.
SEQ ID NO: 6 provides the amino acid sequence of GmBBX53 protein.
SEQ ID NO: 7 provides the consensus amino acid sequence of B-box Zinc Finger domain.
SEQ ID NO: 8 SEQ ID NO: 9 SEQ ID NO: 10 SEQ ID NO: 11 and SEQ ID NO: 12 provide the consensus amino acid sequences of the motifs from the N-terminus to the C-terminus, respectively, after the B-box domain (SEQ ID NO: 7).
SEQ ID NO: 13 provides the amino acid sequence of the B-box domain of AtBBX32.
SEQ ID NO: 14 provides the amino acid sequence of the B-box domain of GmBBX52.
SEQ ID NO: 15 provides the amino acid sequence of the B-box domain of GmBBX53.

### DETAILED DESCRIPTION OF THE INVENTION

The following definitions and methods are provided to better define the present disclosure and to guide those of ordinary skill in the art in the practice of the present disclosure. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1991; and Lewin, Genes V, Oxford University Press: New York, 1994.

This disclosure provides methods for producing a plant that exhibits an enhanced trait selected from the group consisting of altered hexose sugar level, altered starch level, altered sucrose phosphate synthase (SPS) activity, altered ureide level and delayed senescence, as compared to a control plant. More specifically, the method comprises 1) providing a recombinant nucleic acid encoding a polypeptide, wherein the polypeptide comprises a conserved domain having the amino acid sequence of SEQ ID NO: 7; 2) introducing into a plant cell the recombinant nucleic acid to produce a transgenic plant; and 3) growing the plant with an enhanced trait. In another embodiment, the method further comprises the step of selecting a transgenic plant by its ectopic expression of the polypeptide or its enhanced trait, as compared to the control plant. In one aspect of the disclosure, the altered hexose sugar level, altered starch level, altered SPS activity, or altered ureide level is in leaves. In another aspect, the altered hexose sugar level, altered starch level, or altered ureide level is increased hexose sugar level, increased starch level, or increased ureide level in R1 leaves, if the plant is a soybean plant, or in leaves from a plant at a developmental stage equivalent to R1 if the plant is not a soybean plant. In one embodiment, the altered hexose sugar level is increased hexose sugar level in R5 pods, if the plant is a soybean plant, or in pods from a plant at a developmental stage equivalent to R5 if the plant is not a soybean plant. In another embodiment, the altered hexose sugar level or altered ureide level is increased hexose sugar level or increased ureide level in R4 leaves or R5 leaves, if the plant is a soybean plant, or in leaves from a plant at a developmental stage equivalent to R4 or R5 if the plant is not a soybean plant. In another embodiment, the altered level of at least one hexose sugar is decreased level of at least one hexose sugar in R6 leaves, if the plant is a soybean plant, or in leaves from a plant at a developmental stage equivalent to R6 if the plant is not a soybean plant. In yet another embodiment, the altered starch level is increased starch level in R6 leaves, if the plant is a soybean plant, or in leaves from a plant at a developmental stage equivalent to R6 if the plant is not a soybean plant. In still another embodiment, the altered SPS activity is increased SPS activity in R4 leaves or R6 leaves, if the plant is a soybean plant, or in leaves from a plant at a developmental stage equivalent to R4 or R6 if the plant is not a soybean plant. In yet another embodiment, the altered SPS activity is decreased SPS activity in R1 leaves, if the plant is a soybean plant, or in leaves from a plant at a developmental stage equivalent to R1 if the plant is not a soybean plant. In one aspect, the polypeptide of the disclosure further comprises a motif having the amino acid sequence of SEQ ID NO: 8, SEQ ID NO: 9, SEQ ID NO: 10, SEQ ID NO: 11 or SEQ ID NO: 12 at the C-terminus relative to SEQ ID NO: 7. In another aspect, the polypeptide comprises an amino acid sequence selected from the group consisting of a) SEQ ID NO: 4, SEQ ID NO: 5 or SEQ ID NO: 6 and b) a protein sequence at least 80%, 85%, 90%, 91%, 92%, 93%, 94%, 95%, 96%, 97%, 98%, 99% identical to any of SEQ ID NO: 4, SEQ ID NO: 5 or SEQ ID NO: 6. In yet another aspect, the recombinant nucleic acid comprises a nucleic acid sequence selected from the group consisting of a) a nucleic acid sequence comprising SEQ ID NO: 1, SEQ ID NO: 2 or SEQ ID NO: 3; b) a nucleic acid sequence that hybridizes to SEQ ID NO: 1, SEQ ID NO: 2 or SEQ ID NO: 3 under conditions of 1×SSC, and 65° C.; c) a nucleic acid sequence at least 80%, 85%, 90%, 91%, 92%, 93%, 94%, 95%, 96%, 97%, 98%, 99% identical to any of SEQ ID NO: 1, SEQ ID NO: 2 or SEQ ID NO: 3; and d) a complement of any of a)-c). Transgenic cells, plants, plant parts, seeds, progeny plants, products or commodity products produced by this method are aspects of the disclosure, and may be from any plant species, or may be from any crop plant, or may be selected from the group consisting of soybean, cotton, canola, alfalfa, corn, rice, wheat, sunflower, barley, millet, sorghum, sugar beet, sugarcane and vegetables.

The present disclosure further provides a commodity product from the transgenic plant of the disclosure, including, but being not limited to, whole or processed seed, animal feed, oil, meal, flour, mill, flake, bran, protein concentrate, soy protein isolates, hydrolyzed soy protein, biomass and fuel products.

As used herein, a “plant cell” means a plant cell that is transformed with a stably-integrated, non-naturally occurring, recombinant DNA, e.g. by Agrobacterium-mediated transformation or by bombardment using microparticles coated with recombinant DNA or other means. A plant cell of this disclosure can be an originally-transformed plant cell that exists as a microorganism or as a progeny plant cell that is regenerated into a differentiated tissue, e.g. into a transgenic plant with stably-integrated, non-naturally occurring, recombinant DNA, or seed or pollen derived from a progeny transgenic plant.

As used herein, the term “transgene” refers to a polynucleotide molecule artificially incorporated into a host cell's genome. Such transgene may be heterologous to the host cell. As used herein, the term “heterologous” refers to a sequence that is not normally present in a given host genome in the genetic context in which the sequence is currently found. In this respect, the sequence may be native to the host genome, but be rearranged with respect to other genetic sequences within the host sequence.

As used herein, a “transgenic plant” includes a plant, plant part, plant cell or seed whose genome has been altered by the stable integration of recombinant DNA. A transgenic plant includes a plant regenerated from an originally-transformed plant cell and progeny transgenic plants from later generations or crosses of a transformed plant. As a result of such genomic alteration, the transgenic plant is distinctly different from the related wild type plant.

As used herein, a “recombinant DNA molecule” is a DNA molecule comprising a combination of DNA molecules that would not naturally occur together and is the result of human intervention, e.g., a DNA molecule that is comprised of a combination of at least two DNA molecules heterologous to each other, and/or a DNA molecule that is artificially synthesized and comprises a polynucleotide sequence that deviates from the polynucleotide sequence that would normally exist in nature, and/or a DNA molecule that comprises a transgene artificially incorporated into a host cell's genomic DNA and the associated flanking DNA of the host cell's genome. An example of a recombinant DNA molecule is a DNA molecule described herein resulting from the insertion of the transgene into a plant genome, which may ultimately result in the expression of a recombinant RNA and/or protein molecule in that organism. As used herein, the terms “DNA sequence”, “nucleotide sequence” and “polynucleotide sequence” refer to the sequence of nucleotides of a DNA molecule, usually presented from the 5′ (upstream) end to the 3′ (downstream) end. The nomenclature used herein is that required by Title 37 of the United States Code of Federal Regulations §1.822 and set forth in the tables in WIPO Standard ST.25 (1998), Appendix 2, Tables 1 and 3. A polynucleotide may be a nucleic acid, oligonucleotide, nucleotide, or any fragment thereof. In many instances, a polynucleotide comprises a nucleotide sequence encoding a polypeptide (or protein) or a domain or fragment thereof. Additionally, the polynucleotide may comprise a promoter, an intron, an enhancer region, a polyadenylation site, a translation initiation site, 5′ or 3′ untranslated regions, a reporter gene, a selectable marker, or the like.

The polynucleotide can be single-stranded or double-stranded DNA or RNA. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations. The present disclosure is disclosed with reference to only one strand of the two nucleotide sequence strands that are provided in the transgenic plants. Therefore, by implication and derivation, the complementary sequences, also referred to in the art as the complete complement or the reverse complementary sequences, are within the scope of the present disclosure and are therefore also intended to be within the scope of the subject matter claimed.

Both terms “polypeptide” and “protein”, as used herein, mean a polymer composed of two or more amino acids connected by peptide bonds. An amino acid unit in a polypeptide (or protein) is called a residue. The term “amino acid sequence” means the sequence of amino acids in a polypeptide (or protein) that is written starting with the amino-terminal (N-terminal) residue and ending with the carboxyl-terminal (C-terminal) residue. Proteins of the present disclosure are whole proteins or at least a sufficient portion of the protein to impart the relevant biological activity of the protein, e.g. altered hexose sugar level, altered starch level, altered ureide level, altered SPS activity and delayed senescence in transgenic plants as compared to a control plant, as provided by over-expression of AtBBX32, or GmBBX52 or GmBBX53 or a functionally homologous protein. The term “protein” also includes molecules consisting of one or more polypeptide chains. Thus, a polypeptide useful in the present disclosure may constitute an entire gene product or one or more functional portion of a natural protein that provides an enhanced trait of this disclosure.

The term “domain” as used herein refers to a set of amino acids conserved at specific positions along an alignment of sequences of evolutionarily related proteins. While amino acids at other positions can vary between homologs, amino acids that are highly conserved at specific positions indicate amino acids that are likely essential in the structure, stability or function of a protein. Identified by their high degree of conservation in aligned sequences of a family of protein homologs, they can be used as identifiers to determine if any polypeptide in question belongs to a previously identified polypeptide family. Protein domains are identified by querying the amino acid sequence of a protein against Hidden Markov Models that characterize protein family domains (“Pfam domains”) using HMMER software, which is available from the Pfam Consortium. The HMMER software is also disclosed in patent application publication US 2008/0148432 A1 incorporated herein by reference. A protein domain meeting the gathering cutoff for the alignment of a particular Pfam domain is considered to contain the Pfam domain. A conserved domain with respect to presently disclosed polypeptides refers to a domain within a polypeptide family that exhibits a higher degree of sequence homology, such as at least about 56% sequence identity, or at least about 58% sequence identity, or at least about 60% sequence identity, or at least about 65%, or at least about 67%, or at least about 70%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%, amino acid residue sequence identity, to a conserved domain of a polypeptide of the disclosure (e.g., any of SEQ ID NOs: 4-6). Sequences that possess or encode for conserved domains that meet these criteria of percentage identity, and that have comparable biological activity to the present polypeptide sequences, are encompassed by the disclosure.

The term “motif” or “consensus sequence” refers to a short conserved region in the sequence of evolutionarily related proteins. Motifs are frequently highly conserved parts of domains, but may also include only part of the domain, or be located outside of conserved domain (if all of the amino acids of the motif fall outside of a defined domain).

“Percent identity” describes the extent to which the sequences of DNA or protein segments are invariant in an alignment of sequences, for example nucleotide sequences or amino acid sequences. An alignment of sequences is created by manually aligning two sequences, e.g. a stated sequence, as provided herein, as a reference, and another sequence, to produce the highest number of matching elements, e.g. individual nucleotides or amino acids, while allowing for the introduction of gaps into either sequence. An “identity fraction” for a sequence aligned with a reference sequence is the number of matching elements, divided by the full length of the reference sequence, not including gaps introduced by the alignment process into the reference sequence. “Percent identity” (“% identity”) as used herein is the identity fraction times 100.

“Complementary” refers to the natural hydrogen bonding by base pairing between purines and pyrimidines. For example, the sequence A-C-G-T (5′->3′) forms hydrogen bonds with its complements A-C-G-T (5′->3′) or A-C-G-U (5′->3′). Two single-stranded molecules may be considered “partially complementary”, if only some of the nucleotides bond, or “completely complementary” if all of the nucleotides bond. The degree of complementarity between nucleic acid strands affects the efficiency and strength of hybridization and amplification reactions. “Fully complementary” refers to the case where bonding occurs between every base pair and its complement in a pair of sequences, and the two sequences have the same number of nucleotides. The terms “highly stringent” or “highly stringent condition” refer to conditions that permit hybridization of DNA strands whose sequences are highly complementary, wherein these same conditions exclude hybridization of significantly mismatched DNAs. Polynucleotide sequences capable of hybridizing under stringent conditions with the polynucleotides of the present disclosure may be, for example, variants of the disclosed polynucleotide sequences, including allelic or splice variants, or sequences that encode orthologs or paralogs of presently disclosed polypeptides. Nucleic acid hybridization methods are disclosed in detail by Kashima et al. (1985), Sambrook et al. (1989), and by Haymes et al. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Thus, similar nucleic acid sequences from a variety of sources, such as within a plant's genome (as in the case of paralogs) or from another plant (as in the case of orthologs) that may perform similar functions can be isolated on the basis of their ability to hybridize with known related polynucleotide sequences. Numerous variations are possible in the conditions and means by which nucleic acid hybridization can be performed to isolate related polynucleotide sequences having similarity to sequences known in the art and are not limited to those explicitly disclosed herein. Such an approach may be used to isolate polynucleotide sequences having various degrees of similarity with disclosed polynucleotide sequences, such as, for example, those encoding proteins having 56% or greater identity with the conserved domains of disclosed sequences.

As used herein “expressed” means produced, e.g. a protein is expressed in a plant cell when its cognate DNA is transcribed to mRNA that is translated to the protein.

The term “over-expression” as used herein refers to a greater expression level of a gene in a plant, plant cell or plant tissue, compared to expression in a wild-type plant, cell or tissue, at any developmental or temporal stage for the gene. Over-expression can occur when, for example, the genes encoding one or more polypeptides are under the control of a strong promoter (e.g., the cauliflower mosaic virus 35S transcription initiation region). Over-expression may also under the control of an inducible or tissue specific promoter. Thus, over-expression may occur throughout a plant, in specific tissues of the plant, or in the presence or absence of particular environmental signals, depending on the promoter used. Over-expression may take place in plant cells normally lacking expression of polypeptides functionally equivalent or identical to the present polypeptides. Over-expression may also occur in plant cells where endogenous expression of the present polypeptides or functionally equivalent molecules normally occurs, but such normal expression is at a lower level. Over-expression thus results in a greater than normal production, or “over-production” of the polypeptide in the plant, cell or tissue.

As used herein a “control plant” means a plant that does not contain the recombinant DNA that imparts an enhanced trait. A control plant is used to identify and select a transgenic plant that has an enhanced trait. A suitable control plant can be a non-transgenic plant of the parental line used to generate a transgenic plant, i.e. devoid of recombinant DNA. Such a control plant is also referred to as a wild type (wt) plant. A suitable control plant may in some cases be a progeny of a hemizygous transgenic plant line that does not contain the recombinant DNA, known as a negative segregant or negative isoline.

As used herein the term “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, sugar or oil content of seed or leaves, or measuring the activity of enzymes, or by observation of a metabolic or physiological process, e.g. by measuring tolerance to water deprivation or particular salt or sugar concentrations, or by the observation of the expression level of a gene or genes, e.g., by employing Northern analysis, RT-PCR, microarray gene expression assays, or reporter gene expression systems, or by agricultural observations such as hyperosmotic stress tolerance or yield. Any technique can be used to measure the amount of, comparative level of, or difference in any selected chemical compound or macromolecule in the transgenic plants. As used herein an “enhanced trait” means a trait of a transgenic plant that includes, but is not limited to, an enhanced agronomic trait characterized by enhanced plant morphology, physiology, growth and development, yield, nutritional enhancement, disease or pest resistance, or environmental or chemical tolerance. Some aspects of this disclosure include enhanced traits selected from the group consisting of altered hexose sugar level, altered starch level, altered ureide level, altered SPS activity and delayed senescence.

As used herein, the term “hexose sugar” refers to a monosaccharide with six carbon atoms, having the chemical formula C₆H₁₂O₆. Examples of hexose sugar include, but are not limited to, glucose and fructose. The term “hexose sugar” as used herein also includes modified hexose sugars, such as hexose sugar phosphates and uridine diphosphate (UDP) hexose sugars. Examples of hexose sugar phosphates include, but are not limited to, fructose-6-phosphate and glucose-6-phosphate. Examples of uridine diphosphate hexose sugar include, but are not limited to, UDP-glucose.

As used herein, the term “ureides” refers to allantoin and allantoic acid, which are the major forms of nitrogen transported from nodules to the aerial portion of the plant in soybean.

As used herein, “senescence” refers to the process that occurs in a leaf near the end of its active life that is associated with the decrease in chlorophyll, therefore, loss of the green color and the ability of the plant to photosynthesize. “Delayed senescence” as used herein refers to slowing down or delaying of the senescence process, or altered lengths of different reproductive stages compared to control plants, due to the effect of the transgene insertion into the transgenic plants, causing the transgenic plants remaining/staying green after the control plants in the field have turned brown or showed signs of senescence. The delayed senescence may be due to a delay in the onset of the senescence process. There may or may not then be a subsequent acceleration in the progression of senescence once it is started so that the transgenic plants reach full senescence either later than controls or at a relatively similar time as a control plant. The transgenic plants may have the same or similar onset of senescence, but progress at a much slower pace compared to a control plant. Delayed senescence as used herein may also refer to altered lengths/onset of different reproductive stages prior to senescence. This may lead to an overall developmental delay in the transgenic plant, thus resulting in a delay in the entry into senescence. Delayed senescence may also be a combination of what are mentioned above. Delayed senescence may result in plants with leaves visually remaining green for an extended period of time, which may be referred to as “delayed leaf senescence”, or “stay green”. The term “stay green” as used herein encompasses both retention of green leaf tissue (“visual stay green”) and/or the maintenance of photosynthetic activity (“functional stay green”). “Senescence” or “delayed senescence” or “delayed leaf senescence” or “functional stay green” or “stay green” may be assessed by different assays, such as measuring the activities of gene expression, proteins or membrane ions, or by directly measuring photosynthetic activity, or chlorophyll fluorescence. Alternatively, senescence may be assessed by visual inspection of leaf greenness.

As used herein “R1”, “R4”, “R5” and “R6” refer to stages of soybean reproductive development. “R1” is the stage of beginning bloom, where the plants have one or more open flower at any node on the main stem. A node is the part of the stem where a leaf/flower/pod is (or has been) attached. “R4” is the stage of full pod, where the pod is three-quarters of an inch long at one of the four uppermost nodes on the main stem with a fully developed leaf. “R5” is the stage of beginning seed. Seed filing during this stage requires much water and nutrients from the plant. This stage has seed at least ⅛ inch long in one of the pods on one of the four upper nodes of the main stem. “R6” is also known as the “green bean” stage or beginning full seed stage. Total pod weight will peak during this stage. This stage initiates with a pod containing a green seed that fills the pod cavity on at least one of the four top nodes of the main stem. Rapid leaf yellowing will begin right after this stage until R8, or all leaves have fallen.

As used herein a “plant” includes whole plant, transgenic plant, shoot organs/structures (for example, leaves, stems and tubers), roots, flowers and floral organs/structures (for example, bracts, sepals, petals, stamens, carpels, anthers and ovules), seeds (including embryos, endosperm, and seed coat) and fruit (the mature ovary), plant tissues (for example, vascular tissues, ground tissues, and the like) and cells (for example, guard cells, egg cells, pollen, mesophyll cell, and the like), and progeny of same.

As used herein, a “plant part” refers to any part of a plant that is comprised of material derived from a transgenic plant of the present disclosure. Plant parts include but are not limited to cell, pollen, ovule, pod, flower, root or stem tissue, fibers and leaf. Plant parts may be viable, nonviable, regenerable, and/or non-regenerable.

The present disclosure provides a commodity product that is derived from a transgenic plant of the present disclosure. As used herein, a “commodity product” refers to any composition or product that is comprised of material derived from a plant, seed, plant cell, or plant part of the present disclosure. Commodity products may be sold to consumers and may be viable or nonviable. Nonviable commodity products include, but are not limited to, nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, mill, flakes, bran, fiber, and any other food for human consumption; and biomasses and fuel products. Processed soybeans are the largest source of protein feed and vegetable oil in the world. Viable commodity products include, but are not limited to, seeds and plant cells.

Recombinant DNA constructs are assembled using methods well known to persons of ordinary skill in the art and typically comprise a promoter operably linked to DNA, the expression of which provides an enhanced trait. Other construct components may include additional regulatory elements, such as 5′ leaders and introns for enhancing transcription, 3′ untranslated regions (such as polyadenylation signals and sites), DNA for transit or signal peptides.

Numerous promoters that are active in plant cells have been described in the literature. These include promoters present in plant genomes as well as promoters from other sources, including nopaline synthase (NOS) promoter and octopine synthase (OCS) promoters carried on tumor-inducing plasmids of Agrobacterium tumefaciens and the CaMV35S promoters from the cauliflower mosaic virus as disclosed in U.S. Pat. Nos. 5,164,316 and 5,322,938. Useful promoters derived from plant genes are found in U.S. Pat. No. 5,641,876, which discloses a rice actin promoter, U.S. Pat. No. 7,151,204, which discloses a maize chloroplast aldolase promoter and a maize aldolase (FDA) promoter, and US Patent Application Publication 2003/0131377 A1, which discloses a maize nicotianamine synthase promoter. These and numerous other promoters that function in plant cells are known to those skilled in the art and available for use in recombinant polynucleotides of the present disclosure to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene intron. See also US Patent Application Publication 2002/0192813A1, which discloses 5′, 3′ and intron elements useful in the design of effective plant expression vectors.

Recombinant DNA constructs useful in this disclosure will also generally include a 3′ element that typically contains a polyadenylation signal and site. Well-known 3′ elements include those from Agrobacterium tumefaciens genes such as nos 3′, tml 3′, tmr 3′, tms 3′, ocs 3′, tr7 3′, for example disclosed in U.S. Pat. Nos. 6,090,627; 3′ elements from plant genes such as wheat (Triticum aesevitum) heat shock protein 17 (Hsp17 3′), a wheat ubiquitin gene, a wheat fructose-1,6-biphosphatase gene, a rice glutelin gene, a rice lactate dehydrogenase gene and a rice beta-tubulin gene, all of which are disclosed in US Patent Application Publication 2002/0192813 A1; and the pea (Pisum sativum) ribulose biphosphate carboxylase gene (rbs 3′), the 3′ untranslated region from the fiber protein E6 gene of sea-island cotton (Plant Mol. Biol. 30 (2), 297-306 (1996)).

Constructs and vectors may also include a transit peptide for targeting of a gene to a plant organelle, particularly to a chloroplast, leucoplast or other plastid organelle. For descriptions of the use of chloroplast transit peptides see U.S. Pat. No. 5,188,642 and U.S. Pat. No. 5,728,925. For description of the transit peptide region of an Arabidopsis EPSPS gene useful in the present disclosure, see Klee, H. J. et al (MGG (1987) 210:437-442).

Transgenic plants may comprise a stack of one or more polynucleotides disclosed herein with one or more additional polynucleotides resulting in the production of multiple polypeptide sequences. Transgenic plants comprising stacks of polynucleotide sequences can be obtained by either or both of traditional breeding methods or through genetic engineering methods. These methods include, but are not limited to, breeding individual lines each comprising a polynucleotide of interest, transforming a transgenic plant comprising a gene disclosed herein with a subsequent gene, and co-transformation of genes into a single plant cell. Co-transformation of genes can be carried out using single transformation vectors comprising multiple genes or genes carried separately on multiple vectors.

Transgenic plants comprising or derived from plant cells of this disclosure transformed with recombinant DNA can be further enhanced with stacked traits, e.g. a crop plant having an enhanced trait resulting from expression of DNA disclosed herein in combination with herbicide and/or pest resistance traits. For example, genes of the current disclosure can be stacked with other traits of agronomic interest, such as a trait providing herbicide resistance, or insect resistance, such as using a gene from Bacillus thuringensis to provide resistance against lepidopteran, coliopteran, homopteran, hemiopteran, and other insects. Herbicides for which transgenic plant tolerance has been demonstrated and the method of the present disclosure can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175 and a glyphosate-N-acetyl transferase (GAT) disclosed in US Patent Application Publication 2003/0083480 A1 also for imparting glyphosate tolerance; dicamba monooxygenase disclosed in US Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtl) described in Misawa et al, (1993) Plant J. 4:833-840 and in Misawa et al, (1994) Plant J. 6:481-489 for norflurazon tolerance; a polynucleotide molecule encoding acetohydroxyacid synthase (AHAS, aka ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in US Patent Application Publication 2003/010609 A1 for imparting N-amino methyl phosphonic acid tolerance; polynucleotide molecules disclosed in U.S. Pat. No. 6,107,549 for imparting pyridine herbicide resistance. Molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication 2002/0112260. Molecules and methods for imparting insect/nematode/virus resistance are disclosed in U.S. Pat. Nos. 5,250,515; 5,880,275; 6,506,599; 5,986,175 and US Patent Application Publication 2003/0150017 A1.

### Plant Cell Transformation Methods

Numerous methods for transforming chromosomes in a plant cell with recombinant DNA are known in the art and are used in methods of preparing a transgenic plant cell nucleus, cell, and plant. Two effective methods for such transformation are Agrobacterium-mediated transformation and microprojectile bombardment. Microprojectile bombardment methods are illustrated in U.S. Pat. No. 5,015,580 (soybean); U.S. Pat. No. 5,550,318 (corn); U.S. Pat. No. 5,538,880 (corn); U.S. Pat. No. 5,914,451 (soybean); U.S. Pat. No. 6,160,208 (corn); U.S. Pat. No. 6,399,861 (corn); U.S. Pat. No. 6,153,812 (wheat) and U.S. Pat. No. 6,365,807 (rice). Agrobacterium-mediated transformation is described in U.S. Pat. No. 5,159,135 (cotton); U.S. Pat. No. 5,824,877 (soybean); U.S. Pat. No. 5,463,174 (canola); U.S. Pat. No. 5,591,616 (corn); U.S. Pat. No. 5,846,797 (cotton); U.S. Pat. No. 6,384,301 (soybean), U.S. Pat. No. 7,026,528 (wheat) and U.S. Pat. No. 6,329,571 (rice), US Patent Application Publication 2004/0087030 A1 (cotton), and US Patent Application Publication 2001/0042257 A1 (sugar beet), all of which are incorporated herein by reference for enabling the production of transgenic plants. Transformation of plant material is practiced in tissue culture on a nutrient media, i.e. a mixture of nutrients that allows cells to grow in vitro. Recipient targets include, but are not limited to, meristems, hypocotyls, calli, immature embryos, mature embryos and gametic cells such as microspores, pollen, sperm and egg cells. Callus may be initiated from tissue sources including, but not being limited to, immature embryos, mature embryos, hypocotyls, seedling apical meristems, microspores and the like. Cells containing a transgenic nucleus are grown into transgenic plants.

In addition to direct transformation of a plant material with a recombinant DNA, a transgenic plant can be prepared by crossing a first plant comprising a recombinant DNA with a second plant lacking the recombinant DNA. For example, recombinant DNA can be introduced into a first plant cell that is amenable to transformation, which is allowed to grow into a transgenic plant, which can be crossed with a second plant line to introgress the recombinant DNA into the second plant line. A transgenic plant with recombinant DNA providing an enhanced trait, such as increased starch level, can be crossed with a transgenic plant line having other recombinant DNA that confers another trait, for example herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA, e.g. marker identification by analysis for recombinant DNA or, in the case where a selectable marker is linked to the recombinant, by application of the selecting agent such as a herbicide for use with a herbicide tolerance marker, or by selection for the enhanced trait. Progeny plants carrying DNA for both parental traits can be crossed back into the female parent line multiple times, for example usually 6 to 8 generations, to produce a progeny plant with substantially the same genotype as one original transgenic parental line but for the recombinant DNA of the other transgenic parental line.

In the practice of transformation, DNA is typically introduced into only a small percentage of target plant cells in any one transformation experiment. Marker genes are used to provide an efficient system for identification of those cells that are stably transformed by receiving and integrating a recombinant DNA molecule into their genomes. Preferred marker genes provide selective markers that confer resistance to a selective agent, such as an antibiotic or a herbicide. Any of the herbicides to which plants of this disclosure may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (val), hygromycin B (aph IV), spectinomycin (aadA) and gentamycin (aac3 and aacC4) or resistance to herbicides such as glufosinate (bar or pat), dicamba (DMO) and glyphosate (aroA or EPSPS). Examples of such selectable markers are illustrated in U.S. Pat. Nos. 5,550,318; 5,633,435; 5,780,708 and 6,118,047. Markers that provide an ability to visually screen transformants can also be employed, for example, a gene expressing a colored or fluorescent protein such as a luciferase or green fluorescent protein (GFP) or a gene expressing a beta-glucuronidase or uidA gene (GUS) for which various chromogenic substrates are known.

Plant cells that survive exposure to the selective agent, or plant cells that have been scored positive in a screening assay, may be cultured in regeneration media and allowed to mature into plants. Developing plantlets regenerated from transformed plant cells can be transferred to plant growth mix, and hardened off, for example, in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue, selection regimes and plant species. Plants may be pollinated using conventional plant breeding methods known to those of skill in the art and seed produced, for example self-pollination is commonly used with transgenic corn. The regenerated transformed plant or its progeny seed or plants can be tested for expression of the recombinant DNA and selected for the presence of an enhanced agronomic trait.

Selection Methods for Transgenic Plants with Enhanced Traits

Within a population of transgenic plants each regenerated from a plant cell with recombinant DNA, many plants that survive to fertile transgenic plants that produce seeds and progeny plants will not exhibit an enhanced agronomic trait. Selection from the population is necessary to identify one or more transgenic plant that can provide plants with an enhanced trait. Transgenic plants having enhanced traits are selected from populations of plants regenerated or derived from plant cells transformed as described herein by evaluating the plants in a variety of assays to detect an enhanced trait. For efficiency a selection method is designed to evaluate multiple transgenic plants (events) comprising the recombinant DNA, for example multiple plants from 2 to 20 or more transgenic events. These assays also may take many forms including, but not being limited to, direct screening for the trait in a greenhouse or field trial or by screening for a surrogate trait. Such analyses can be directed to detecting changes in, for example, the chemical composition, physiological properties, enzymatic activity or morphology of the plant. Changes in chemical compositions within a plant can be detected for example by analysis of the levels of starch, hexose sugars, or ureides. Changes in enzymatic activity can be detected for example by analysis of the level of enzymatic activity of SPS. Other selection properties include stay green or delayed senescence.

The methods of this disclosure can be applied to any plant cell, plant, seed or pollen, e.g. any fruit, vegetable, grass, tree or ornamental plant, such as corn, soybean, cotton, canola, alfalfa, wheat, rice, sugarcane, sugar beet and vegetable plants.

### EXAMPLES

The following examples are included to demonstrate certain embodiments of the disclosure. It should be appreciated by those of skill in the art that the techniques disclosed in the examples that follow represent techniques discovered by the inventors to function well in the practice of the disclosure. However, those of skill in the art should, in light of the present disclosure, appreciate that many changes can be made in the specific embodiments that are disclosed and still obtain a like or similar result without departing from the spirit and scope of the disclosure, therefore all matter set forth or shown in the examples is to be interpreted as illustrative and not in a limiting sense.

### Example 1

### Identification of Protein Conserved Patterns

This example describes identification of common domains and motifs. Common protein domains were identified by searching genes of the current disclosure against the Pfam database, also known as Pfam-A, which is a collection of protein family alignments that were constructed semi-automatically using Hidden Markov Models (HMMS) (The Pfam protein families database: R. D. Finn et al., Nucleic Acids Research 2010 Database Issue 38:D211-222). Version 23.0 of Pfam-A contains 10,340 families. 73.75% of all proteins in Pfamseq contain a match to at least one Pfam domain. Pfam is based on a sequence database called Pfamseq—Pfamseq 23 is based on UniProt 12.5 (Swiss-Prot 54.5 and SP-TrEMBL 37.5). Pfamseq 23 contains 5,323,441 sequences and 1,738,474,641 residues.

Using the sequences of AtBBX32, GmBBX52 and GmBBX53 to search against the Pfam database with the program hmmpfam, which is part of HMMER package v2.3.2 and the Pfam GA gathering threshold cut-offs, it was shown that one copy of the B-box zinc finger domain is present at the N-terminus of each gene (Table 1).

TABLE 1
Identification of B-box zinc finger domain

| SEQ | Pfam | | | | |
|---|---|---|---|---|---|
| ID NO | domain | From | To | Score | E-value |
| 4 | zf-B-box | 1 | 47 | 39.5 | 1.40E−08 |
| 5 | zf-B-box | 1 | 47 | 45.8 | 1.70E−10 |
| 6 | zf-B-box | 1 | 46 | 22.6 | 0.00061 |

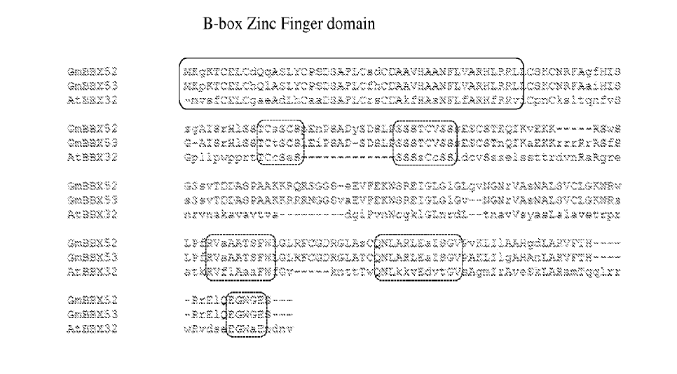

Common motifs were identified by aligning the protein sequences using the program MUSCLE v3.52 with the default parameters, and by manually inspecting multiple sequence alignment and making adjustment as needed. FIG. 1 shows the alignment of the sequences and the common domain/motifs identified. Besides the B-box domain, additional motifs are, in the order of N-terminus to C-terminus: ZF-B_box (SEQ ID NO: 7), TCXSXS (SEQ ID NO: 8), SSSXCXSS (SEQ ID NO: 9), RVX₂AX₂FW (SEQ ID NO: 10), QNLX₃EX₃GV (SEQ ID NO: 11), and EGWXE (SEQ ID NO: 12).

### Example 2

### Transformation of Soybean and Selection of Events with an Enhanced Trait

This example describes transformation and generation of transgenic soybean events and selection of events with an enhanced trait, using construct pMON83132 as an example. The same methods were used for other constructs within the scope of the disclosure.

An Agrobacterium-mediated transformation method was used to transform soybean cells with the binary construct pMON83132. pMON83132 contains two plant transformation cassettes or T-DNAs. Each cassette is flanked by right border and left border sequences. The transgenic insert comprises an expression cassette containing an enhanced 35S promoter from cauliflower mosaic virus (CaMV), operably linked to a DNA molecule encoding the AtBBX32 protein (SEQ ID NO: 4), operably linked to the 3′ untranslated region from fiber protein E6 gene of Gossypium barbadense (cotton). The second transformation cassette contains a chimeric promoter consisting of enhancer sequences from the promoter of the figwort mosaic virus (FMV) 35S RNA combined with the promoter of the elongation factor 1A gene (Tsf1) from Arabidopsis thaliana. It also contains the 5′ untranslated leader, and the intron of the elongation factor 1A gene (Tsf1) from Arabidopsis, operably linked to a DNA molecule encoding a chloroplast transit peptide (CTP2) from Arabidopsis EPSP synthase, fused to a codon modified coding sequence of the aroA gene from the Agrobacterium sp. strain CP4 encoding the CP4 EPSPS protein, operably linked to a 3′ untranslated region of the RbcS2 gene from Pisum sative. The CP4 aroA gene confers tolerance to glyphosate, and was used as a selectable marker. Table 2 is a summary of the genetic elements in pMON83132.

TABLE 2
Summary of the genetic elements in pMON83132.

| | | |
|---|---|---|
| | Location in | |
| Genetic | Construct | |
| Element | pMON83132 | Function (Reference) |

T-DNA I

| | | |
|---|---|---|
| B¹-Left Border | 1-442 | DNA region from Agrobacterium tumefaciens |
| | | containing the left border sequence used for transfer |
| | | of the T-DNA (Barker et al., Plant Mol. Biol. 2: 335- |
| | | 350, 1983). |
| P²-e35S | 511-1123 | Promoter for the cauliflower mosaic virus (CaMV) |
| | | 35S RNA (Odell et al., Nature 313: 810-812, 1985) |
| | | containing the duplicated enhancer region (Kay et al., |
| | | Science 236: 1299-1302, 1987) that directs |
| | | transcription in plant cells. |
| CS³-AtBBX32 | 1148-1825 | Coding sequence of the AtBBX32 gene from |
| | | Arabidopsis thaliana encoding a zinc finger protein |
| | | (B-box type) (Khanna et al., Plant Cell 21: 3416- |
| | | 3420, 2009), which modulates aspects of diurnal |
| | | biology (Holtan et al, submitted). |
| T⁴-E6 | 1840-2154 | 3′ UTR region of the E6 gene of Gossypium |
| | | barbadense (sea-island cotton) that encodes a fiber |
| | | protein involved in early fiber development (John, |
| | | Plant Mol Biol. 30: 297-306, 1996), which functions |
| | | to direct polyadenylation of mRNA. |
| B-Right Border | 2193-2549 | DNA region from Agrobacterium tumefaciens |
| | | containing the right border sequence used for transfer |
| | | of the T-DNA (Depicker et al., J. of Mol. and |
| | | Applied Genetics 1: 561-573, 1982; Zambryski et al., |
| | | J. of Mol. and Applied Genetics 1: 361-370, 1982). |

T-DNA II

| | | |
|---|---|---|
| B-Right Border | 2721-3077 | DNA region from Agrobacterium tumefaciens |
| | | containing the right border sequence used for transfer |
| | | of the T-DNA (Depicker et al., J. of Mol. and |
| | | Applied Genetics 1: 561-573, 1982; Zambryski et al., |
| | | J. of Mol. and Applied Genetics 1: 361-370, 1982). |
| P-FMV/Tsf1 | 3100-4139 | Chimeric promoter consisting of enhancer sequences |
| | | from the promoter of the Figwort Mosaic virus |
| | | (FMV) 35S RNA (Richins et al., Nucleic Acids |
| | | Research 15: 8451-8466, 1987) combined with the |
| | | promoter of the elongation factor 1A gene (Tsf1) |
| | | from Arabidopsis thaliana (Axelos et al., Molecular |
| | | and General Genetics 219: 106-112, 1989). It is |
| | | associated with constitutive expression of the gene. |
| L⁵-Tsf1 | 4140-4185 | The 5′ untranslated leader of the elongation factor 1A |
| | | gene (Tsf1) from Arabidopsis thaliana (Axelos et al., |
| | | Molecular and General Genetics 219: 106-112, 1989), |
| | | which enhances gene expression. |
| I⁶-Tsf1 | 4186-4807 | Intron of the elongation factor 1A gene (Tsf1) from |
| | | Arabidopsis thaliana (Axelos et al., Molecular and |
| | | General Genetics 219: 106-112, 1989), that enhances |
| | | gene expression. |
| TS⁷-CTP2 | 4817-5044 | Targeting sequence of the ShkG gene from |
| | | Arabidopsis thaliana encoding EPSPS containing the |
| | | transit peptide region) that directs transport of the |
| | | EPSPS protein to the chloroplast (Klee et al., |
| | | Molecular and General Genetics 210: 437-442, 1987). |
| CS-modified cp4 | 5045-6412 | Codon modified coding sequence of the aroA gene |
| epsps | | from the Agrobacterium sp. strain CP4 encoding the |
| | | CP4 EPSPS protein (Padgette et al., 1996, in |
| | | Herbicide-Resistant Crops: Agricultural, Economic, |
| | | Environmental, Regulatory, and Technological |
| | | Aspects, S. O. Duke, (ed.), Pages 53-84, CRC Press, |
| | | Boca Raton, FL). |
| T-E9 | 6419-7061 | 3′ nontranslated region of the RbcS2 gene from Pisum |
| | | sativum (pea) encoding the Rubisco small subunit, |
| | | which functions to direct polyadenylation of the |
| | | mRNA (Coruzzi et al., EMBO J. 3: 1671-1679, 1984). |
| B-Left Border | 7076-7486 | DNA region from Agrobacterium tumefaciens |
| | | containing the left border sequence used for transfer |
| | | of the T-DNA (Barker et al., Plant Mol. Biol. 2: 335- |
| | | 350, 1983). |

Vector Backbone

| | | |
|---|---|---|
| aadA | 7582-8470 | Bacterial promoter, coding sequence, and 3′ UTR for |
| | | an aminoglycoside-modifying enzyme, 3″(9)-O- |
| | | nucleotidyltransferase from the transposon Tn7 (Fling |
| | | et al., Nucleic Acids Research 13: 7095-7106, 1985) |
| | | that confers spectinomycin and streptomycin |
| | | resistance. |
| OR⁸-ori-pBR322 | 9001-9589 | Origin of replication from pBR322 for maintenance |
| | | of plasmid in E. coli (Sutcliffe, 1979, Complete |
| | | nucleotide sequence of the Escherichia coli plasmid |
| | | pBR322. Pages 77-90 in Cold Spring Harbor |
| | | Symposia on Quantitative Biology, Cold Spring |
| | | Harbor, NY, Cold Spring Harbor Laboratory Press). |
| CS-rop | 10017-10208 | Coding sequence for repressor of primer protein |
| | | derived from the ColE1 plasmid for maintenance of |
| | | plasmid copy number in E. coli (Giza and Huang, |
| | | Gene 78: 73-84, 1989). |
| OR-ori V | 10946-11342 | Origin of replication from the broad host range |
| | | plasmid RK2 for maintenance of plasmid in |
| | | Agrobacterium (Stalker et al., Molecular and General |
| | | Genetics 181: 8-12, 1981). |

¹B, Border
²P, Promoter
³CS, Coding Sequence
⁴T, Transcription Termination Sequence
⁵L, Leader
⁶I, Intron
⁷TS, Targeting Sequence
⁸OR, Origin of Replication

After transformation with construct pMON83132, transformed cells were allowed to grow and multiply on media containing glyphosate. Plants were regenerated from surviving cells. Hundreds of transformation events were produced. After molecular screening and linkage Southern analysis, events with undesirable molecular traits were eliminated, such as presence of multiple copies of the transgene and/or molecular complexity of the insert, the presence of the transformation vector backbone sequence and linkage of the AtBBX32 cassette to the CP4 cassette.

Further screening and characterization of the remaining events at the R1 and R2 generations resulted in 68 events that were CP4 marker-free, Agrobacterium Ti plasmid backbone-free, contained only single copy of the transgene with desirable expression levels, and with no undesirable agronomic phenotypes. These events were carried forward for year 1 field test in the US. Following further evaluation and selection for improved trait such as increased yield, selected events were advanced for further testing and evaluation.

### Example 3

### Analysis of Levels of Hexose Sugars, Starch and Ureides in AtBBX32 Transgenic Soybean Plants

This example describes targeted metabolite analysis of AtBBX32 transgenic soybean events for altered hexose sugar levels, altered starch levels and altered ureide levels.

Since carbon and nitrogen assimilation and distribution are both closely linked to the circadian clock and because perturbations of these pathways may lead to subtle physiological changes resulting in enhanced source capacity, experiments were performed to analyze the abundance of targeted metabolites involved in primary carbon and nitrogen metabolism in transgenic soybean plants over-expressing AtBBX32.

Tissue samples were harvested from randomly selected field grown soybean plants at R1 (an early reproductive stage where soy first begins to flower) or R6 stage (the late reproductive stage prior to senescence and near the end of the critical period for seed fill) at three time points for metabolite analysis, including one hour before dawn, one hour after dawn, and 9 hours after dawn. In a separate experiment, leaf and pod tissues were harvested similarly from randomly selected field grown soybean plants at R4 stage (full pod stage) at 6 different time points: 1 hr pre-dawn, 1 hr, 9 hr, 16 hr, 18 hr and 20 hr post dawn. Apex and source leaf were removed at R1 stage, while source leaf only was sampled at the R4 and R6 stages. Apical tissue was defined as the top quarter inch of the stem, not including newly emerged trifoliates. Source leaf was defined as the fourth fully expanded trifoliate from the top of the plant (dark green, large in size). Three plants were pooled per sample and three bio-replicates were collected from each plot replicate, so that a total of 9 plants were sampled per plot. Samples were immediately placed in liquid nitrogen for flash freezing and transferred to dry ice post-harvest for transport. Samples were stored at −80° C. prior to processing.

The LC-MS/MS methods described by Harrigan et al (G. G. Harrigan, et al., Agric. Food Chem. 55 (2007) 6177-6185) were used to detect soluble sugars. The extraction and the LC/MS method for detecting ureides (allantoin and allantoic acid) were modified from the method published in Ohtake et al. (N. Ohtake, et al., Soil Sci. Plant Nutr. 50 (2004) 241-248). Starch levels in dry powdered leaf tissue were measured enzymatically after first removing the soluble simple sugars with an 80% ethanol extraction. Precipitated starch was hydrolyzed enzymatically into glucose monomers, which were quantified spectrophotometrically using a coupled enzyme assay of hexokinase and Glucose-6-Phosphate Dehydrogenase.

### Hexose Sugar Levels

Comparison of hexose sugar levels between transgenic soybean plants and the wild-type or the negative segregant control plants showed that the transgenic soybean plants exhibited, in R1 source leaf tissues, higher levels of glucose and/or fructose, members of the sucrose sugar family, at 1 hour and/or 9 hours post-dawn (Table 3). Hexose sugar levels were also altered in R6 leaf tissues, where they were decreased. However, the only decreases that were statistically significant were in fructose when the transgenic soybean plants were compared to the negative segregant.

TABLE 3
Hexose sugar changes in R1 and R6 leaves of AtBBX32
transgenic plants compared to controls

| | | | Fructose | Glucose |
|---|---|---|---|---|
| | Control | Time point | (% change) | ((% change) |
| R1 | WT | 1 hr pre-dawn | −3.20 | 14.29 |
| leaves | | 1 hr post-dawn | 20.75 | 29.05** |
| | | 9 hr post-dawn | 30.19** | 40.61** |
| | Negative | 1 hr pre-dawn | −20.91* | −20.11 |
| | segregant | 1 hr post-dawn | 22.25 | 23.76* |
| | | 9 hr post-dawn | 22.63** | 24.39* |
| R6 | WT | 1 hr pre-dawn | −16.53 | −4.69 |
| leaves | | 1 hr post-dawn | −26.30 | −12.20 |
| | | 9 hr post-dawn | −23.14 | −7.27 |
| | Negative | 1 hr pre-dawn | −28.95* | −12.02 |
| | segregant | 1 hr post-dawn | −33.09* | −14.23 |
| | | 9 hr post-dawn | −31.56* | −13.60 |

*p < 0.1
*p < 0.05

In a separate experiment, R4 leaf tissues were collected similarly, but at the following time points: 1 hr pre-dawn, 1 hr, 9 hr, 16 hr, 18 hr and 20 hr post dawn. Higher levels of glucose and/or fructose were observed at most time points when compared to either the wild-type control or the negative segregant control plants (Table 4).

TABLE 4
Hexose sugar changes in R4 leaves of AtBBX32
transgenic plants compared to controls

R4 leaves

| | | Fructose | Glucose |
|---|---|---|---|
| Control | Time point | (% change) | (% change) |
| WT | 1 hr pre-dawn | 34.50** | 47.66** |
| | 1 hr post-dawn | 7.4 | 18.08 |
| | 9 hr post-dawn | 14.72 | 33.94* |
| | 16 hr post-dawn | 40.70** | 56.62** |
| | 18 hr post-dawn | 23.01* | 26.63** |
| | 20 hr post-dawn | 28.39 | 47.87** |
| Negative | 1 hr pre-dawn | 36.87** | 50.73** |
| segregant | 1 hr post-dawn | 12.34 | 30.49* |
| | 9 hr post-dawn | 24.20** | 38.01* |
| | 16 hr post-dawn | 51.74** | 53.12** |
| | 18 hr post-dawn | 26.95* | 29.60** |
| | 20 hr post-dawn | 40.29* | 56.68** |

*p < 0.1
**p < 0.05

### Starch Levels

Analyses of starch levels revealed that AtBBX32 transgenic plants exhibited altered starch levels at various stages in reproductive development. During early reproductive development (R1 stage), starch levels were significantly increased at 1 and/or 9 hours post-dawn in R1 leaves. Later in development (R6 stage), starch levels were significantly higher at 1 hour pre-dawn compared to the levels in the wild-type plants (Table 5). Similar results were obtained when compared to the negative segregant plants (Table 5). The increase in starch (source compound) is consistent with altered utilization of carbohydrate reserves during the night, the capacity to anticipate dawn and the maintenance of plant productivity (Graf et al., Proc. Natl. Acad. Sci. 107 (20) 9458-9463, 2010).

TABLE 5
Starch changes in R1 and R6 leaves of AtBBX32
transgenic plants compared to controls

| | | Starch (% change) | |
|---|---|---|---|
| | | Comparison to | Comparison to |
| Stage/Tissue | Time point | WT control | negative segregant |
| R1 | 1 hr pre-dawn | 24.96 | 13.19 |
| leaves | 1 hr post-dawn | 56.92** | 39.96* |
| | 9 hr post-dawn | 11.15** | 7.18 |
| R6 | 1 hr pre-dawn | 26.06** | 22.46** |
| leaves | 1 hr post-dawn | 11.64 | 24.08** |
| | 9 hr post-dawn | 12.71 | 14.88* |

*p < 0.1
**p < 0.05

### Ureide Levels

Altered levels of the ureide compounds, allantoin and allantoic acid, were also observed in AtBBX32 transgenic plants compared to the wild type control plants at most time points tested. For example, significant changes were observed at 1 hr pre-dawn and 9 hr post-dawn in R1 leaf tissues (Table 6). When compared to the negative segregant plants, AtBBX32 transgenic plants showed significantly increased levels of allantoin at 1 hr post-dawn and 9 hr post-dawn in R1 leaf tissues, whereas increased levels of allantoic acid were observed at 1 hr pre-dawn. Similarly, in another experiment, significant increase in allantoic acid level was observed in R4 leaves at 18 hr post-dawn when compared to the WT control, and at 18 hr and 20 hr post-dawn when compared to the negative segregant. These compounds are produced in the nodules of soybean by N-fixing bacteria and are transported to the leaf to serve as the building blocks for amino acids and other nitrogenous compounds.

TABLE 6
Ureide changes in AtBBX32 transgenic
plants compared to controls

| Stage/ | | | Allantoin | Allantoic acid |
|---|---|---|---|---|
| Tissue | Control | Time point | (% change) | (% change) |
| R1 | WT | 1 hr pre-dawn | 89.75** | 109.82** |
| leaves | | 1 hr post-dawn | −1.08 | 43.48* |
| | | 9 hr post-dawn | 98.15** | 206.87** |
| | Negative | 1 hr pre-dawn | 57.36* | 29.12 |
| | segregant | 1 hr post-dawn | 26.78 | 82.35** |
| | | 9 hr post-dawn | 28.6 | 131.38** |
| R6 | WT | 1 hr pre-dawn | 135.48 | 38.23 |
| leaves | | 1 hr post-dawn | 88.71 | 54.41 |
| | | 9 hr post-dawn | 71.76 | 75.07 |
| | Negative | 1 hr pre-dawn | 20.19 | −29.11 |
| | segregant | 1 hr post-dawn | −19.25 | −19.75 |
| | | 9 hr post-dawn | −2.7 | −7.85 |
| R4 | WT | 1 hr pre-dawn | 16.26 | −1.39 |
| leaves | | 1 hr post-dawn | 2.54 | 0.32 |
| | | 9 hr post-dawn | 7.42 | −2.39 |
| | | 16 hr post-dawn | −0.28 | 3.81 |
| | | 18 hr post-dawn | 7.91 | 26.53** |
| | | 20 hr post-dawn | −19.9 | 12.58 |
| | Negative | 1 hr pre-dawn | −2.3 | −8.43 |
| | segregant | 1 hr post-dawn | 8.15 | 9.93 |
| | | 9 hr post-dawn | −6.16 | −10.12 |
| | | 16 hr post-dawn | 14.09 | 1.7 |
| | | 18 hr post-dawn | −7.4 | 18.73* |
| | | 20 hr post-dawn | 15.59 | 23.34** |

*p < 0.1
**p < 0.05

### Example 4

### Sucrose Phosphate Synthase Activity

This example describes analysis of the enzymatic activity of sucrose phosphate synthase (SPS) in transgenic soybean events.

Since changes were observed in hexose sugar and starch levels in AtBBX32 transgenic plants, and the fact that metabolism is ultimately regulated at the level of individual enzymes, experiments were performed to test the activities of several enzymes involved in key metabolic processes from primary metabolism in AtBBX32 transgenic and both wild type and negative segregant control samples. In vitro assays were performed with soluble protein extracts prepared from the same R1, R4 and R6 source leaf tissue as described in Example 2. SPS activity is defined as nmols of sucrose produced from UDP-glucose and D-fructose 6-phosphate per minute per mg of protein. Total protein levels of the samples were measured using the Bradford Reagent from BioRad. SPS activity was increased significantly in R6 source leaves from the transgenic plants (Table 7), although a percent increase over the wild type control could not be calculated because SPS activity in the wild type control was lower than the limit of detection. On the other hand, SPS activity was significantly decreased in R1 leaves of AtBBX32 transgenic plants at 1 hr and 9 hr post-dawn when compared to the WT control. In a separate experiment, increased SPS activity was also observed in R4 leaves at 9 hr post-dawn when compared to the WT control, and at 16 hr and 20 hr post-dawn when compared to the negative segregant.

Although an increase in the activity of sucrose phosphate synthase was observed in R6 leaves at all time points tested in transgenic plants compared to the negative segregant control plants and to the wild type control plants, suggesting increased sucrose production, sucrose did not accumulate in source leaf tissue, perhaps due to its active transport to developing grain.

TABLE 7
SPS activity in AtBBX32 transgenic
events compared control plants

| | | | SPS activity |
|---|---|---|---|
| Stage | Control | Time point | (% change) |
| R1 | WT | 1 hr pre-dawn | −14.29 |
| leaves | | 1 hr post-dawn | −23.49** |
| | | 9 hr post-dawn | −25.72* |
| | Negative | 1 hr pre-dawn | −12.18 |
| | segregant | 1 hr post-dawn | −0.15 |
| | | 9 hr post-dawn | −14.50 |
| R6 | WT | 1 hr pre-dawn | ND |
| leaves | | 1 hr post-dawn | ND |
| | | 9 hr post-dawn | ND |
| | Negative | 1 hr pre-dawn | 55.56** |
| | segregant | 1 hr post-dawn | 236.91** |
| | | 9 hr post-dawn | 51.59** |
| R4 | WT | 1 hr pre-dawn | −2.53 |
| leaves | | 1 hr post-dawn | −4.58 |
| | | 9 hr post-dawn | 18.56* |
| | | 16 hr post-dawn | 21.52 |
| | | 18 hr post-dawn | −3.54 |
| | | 20 hr post-dawn | 19.24 |
| | Negative | 1 hr pre-dawn | 14.74 |
| | segregant | 1 hr post-dawn | −5.74 |
| | | 9 hr post-dawn | 12.27 |
| | | 16 hr post-dawn | 28.00* |
| | | 18 hr post-dawn | 5.01 |
| | | 20 hr post-dawn | 25.90* |

*p < 0.1
**p < 0.05

### Example 5

### Delayed Senescence

Plant senescence was assessed by visual inspection of leaf greenness. Senescence was scored on a scale of 1-6 (1=no senescence: no leaf showing visible yellowing in the plot; 6=full senescence: all leaves are yellow and fully senesced in the plot) for a three week period beginning at the onset of senescence in control lines until the end of the season. The scores were entered into a handheld Symbol MC-70 data recorder.

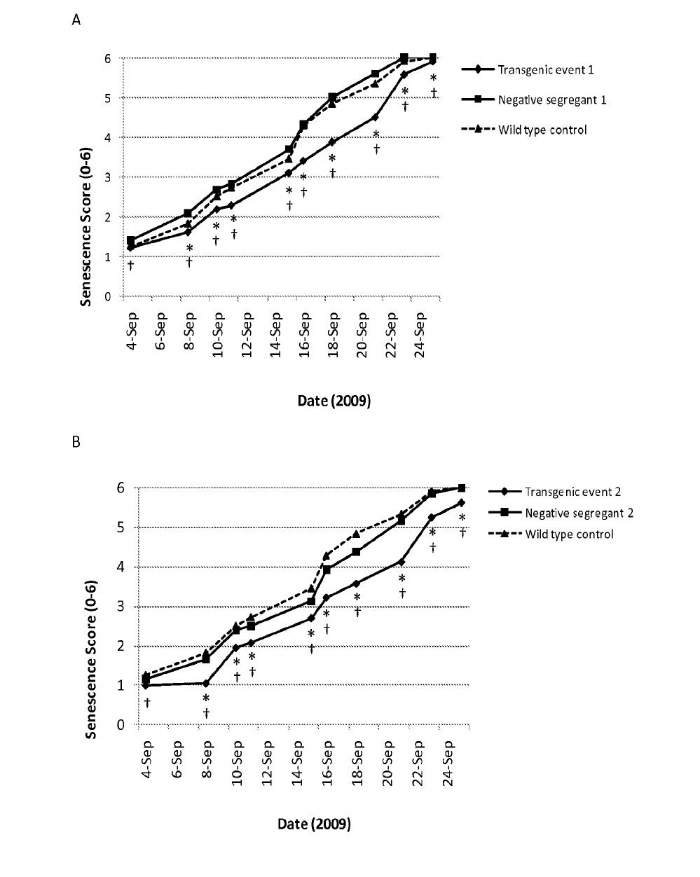

It was observed that AtBBX32 transgenic plants showed a delay in leaf senescence compared to either the negative segregant or the wild type control plants (FIG. 2). Senescence proceeded relatively slowly in the transgenic lines during the first two and a half weeks of monitoring, but accelerated towards the very end of the season, reaching full senescence two days later than did the negative segregant or the wild type controls. These data suggest that the transgenic lines retained greater photosynthetic capacity than did control lines, but that the date of final maturity of the plants was delayed by only two days.

### Example 6

### Analysis of Levels of Hexose Sugars and Ureides in GmBBX 52 Transgenic Soybean Plants and GmBBX53 Transgenic Soybean Plants

This example describes analysis of GmBBX52 transgenic soybean events and GmBBX53 transgenic soybean events for levels of hexose sugars and ureides in R5 leaves and pods.

Similar experiments were conducted as described in Example 3 to measure several key metabolite levels in transgenic soybean plants containing GmBBX53 and GmBBX52. As shown in Table 8, R5 leaf and pod samples from 3 events growing under field conditions were collected and analyzed for GmBBX52 transgenic plants. Similarly, R5 leaf and pod samples from 4 events growing in field conditions were collected and analyzed for GmBBX53 transgenic plants. In a separate experiment, R5 leaf samples from 6 events of AtBBX32 overexpressing plants were harvested and analyzed. Similar results were obtained for GmBBX52 and GmBBX53 transgenic plants when compared to AtBBX32 transgenic plants: events from all three constructs exhibited increased levels of hexose sugars and ureides in R5 leaves. Furthermore, increased levels of hexose sugars were also demonstrated in R5 pods of GmBBX52 transgenic plants and GmBBX53 transgenic plants (Table 8).

TABLE 8
Hexose sugar and ureide levels in transgenic soybean plants

| | Gene Name | | | | |
|---|---|---|---|---|---|
| | AtBBX32 | GmBBX52 | GmBBX52 | GmBBX53 | GmBBX53 |
| Construct | pMON83132 | pMON108080 | pMON108080 | pMON98939 | pMON98939 |
| # Events Tested | 6 | 3 | 3 | 4 | 4 |
| Stage | R5 | R5 | R5 | R5 | R5 |
| Tissue | Leaf | Leaf | Pod | Leaf | Pod |
| UDP-Glucose | ↑ | ↑ | ↑ | ↑ | ↑ |
| Fructose | ↑ | ↑ | ↑ | ↑ | ↑ |
| Glucose | ↑ | ↑ | ↑ | ↑ | ↑ |
| Ureides | ↑ | ↑ | neutral | ↑ | neutral |

↑ denotes an increase in the level

### Example 7

### Transformation of Cotton (Gossypium herbaceum) and Selection of Events with an Enhanced Trait

This example illustrates transformation and generation of transgenic cotton plants, and selection of events with an enhanced trait.

Transgenic cotton plants comprising a polynucleotide comprising SEQ ID NO:7 were created through Agrobacterium-mediated transformation of cotton hypocotyl tissue utilizing a plant transformation vector comprising the expression cassette similar to the one described in Example 2. Methods for transforming cotton are known in the art. Following selection and regeneration, individual transgenic cotton plantlets were obtained. Rooted plants with normal phenotypic characteristics were selected and transferred to soil for growth and further assessment

After characterization to eliminate events with undesirable characteristics, a few events were carried forward for field yield testing and other studies.

### Example 8

### Analysis of Levels of Hexose Sugars in AtBBX32 Transgenic Cotton Plants

This example describes analysis of AtBBX32 transgenic cotton events for increased levels of hexose sugars as compared to a control.

Hexose sugar levels were also analyzed in cotton transgenic plants comprising a polynucleotide comprising SEQ ID NO:7 using similar method as described in Examples 3 and 4. Briefly, leaf samples were collected at 2 different time points from green house grown cotton plants at full bloom stage, which is roughly equivalent to the R3/R4 stages in soybean. Each treatment contained 3-6 replicates. Of the 3 events tested, one showed increased levels of hexose sugars or modified sugars, such as UDP-glucose, fructose, fructose-6 phosphate (fructose-6-P) and glucose-6-phosphate (glucose-6-P) (Table 9). The same event also showed increased yield under stress and broad acre yield conditions. On the other hand, the other two events did not demonstrate increased levels of hexose sugars in the tissue samples tested. PCR analysis revealed loss of the SEQ ID NO:7 polynucleotide in one of those two transgenic events.

TABLE 9
Hexose sugar levels in transgenic cotton
plants comprising SEQ ID NO: 7

| Gene Name | Event 1 | Event 2 | Event 3 |
|---|---|---|---|
| UDP-Glucose | — | ↑* | — |
| Fructose | — | ↑ | — |
| Glucose-6-P | — | ↑ | — |
| Fructose-6-P | — | ↑ | ↓ |

“↑” or “↓” denotes an increase or a decrease in the level
*denotes p < 0.1

### Example 9

### Transformation of Canola (Brassica napus) and Selection of Events with an Enhanced Trait

This example illustrates transformation and generation of transgenic canola plants, and selection of events with an enhanced trait.

Transgenic canola plants overexpressing AtBBX32 were created through Agrobacterium-mediated transformation of explants from Brassica napus utilizing a plant transformation vector comprising the expression cassette similar to the one described in Example 2. Methods for transforming canola are known in the art. Transformed cells were then selected on media containing a selection agent and surviving cells were regenerated into plants.

Many events were generated. After phenotypic screening and molecular characterizations similar to the ones described in Example 2, a few selected events were advanced to field yield testing and other studies.

### Example 10

### Analysis of Levels of Hexose Sugars in AtBBX32 Transgenic Canola Plants

This example describes analysis of AtBBX32 transgenic canola events for increased levels of hexose sugars as compared to a control.

Hexose sugar levels were also analyzed in canola transgenic plants containing AtBBX32 using similar method as described in Examples 3 and 4. Briefly, different tissue samples were collected from 2 events, along with their negative segregant controls, from growth chamber grown canola plants at different development stages at one time-point (1:30 pm). Each treatment contained 5 replicates. Stage 18 refers to the leaf development stage, where the plants has 8 leaves unfolded; stage 55 refers to the inflorescence emergence stage, where the plants have bolted, individual flower buds (on main inflorescence) are visible but are still closed; stage 72 refers to the seed development stage, where 20% of the pods reach their final size. Stages 18, 55 and 72 in canola are roughly equivalent to the V6, R1 and R6 stages in soybean. Table 10 shows levels of hexose sugars in the source leaves of two transgenic canola events compared to their negative segregant controls. Hexose sugar levels were also tested in ripening pods at seed ripening stage, where 20% of the seeds have fully matured and are black and hard (Table 11).

TABLE 10
Hexose sugar levels in source leaves of transgenic canola plants

| Stage | Hexose sugar | Event 1 | Event 2 |
|---|---|---|---|
| 18 | Fructose | ↑ | ↑ |
| | Glucose | ↓ | ↑ |
| 55 | Fructose | ↑* | ↑ |
| | Glucose | ↑ | ↑ |
| 72 | Fructose | ↑ | ↓ |
| | Glucose | ↑ | ↓** |

*denotes p < 0.1
**denotes p < 0.05

TABLE 11
Hexose sugar levels in ripening pods of transgenic canola plants

| Hexose sugar | Event 1 | Event 2 |
|---|---|---|
| Fructose | ↑ | ↑* |
| Mannose | ↑ | ↑* |
| UDP-Glucose | ↑ | ↑** |
| Galactose | ↑ | ↑ |

*denotes p < 0.1
**denotes p < 0.05

We claim:

1. A method for producing a plant that exhibits an enhanced trait selected from the group consisting of altered levels of at least one hexose sugar, altered starch levels, altered sucrose phosphate synthase activity, altered levels of at least one ureide and delayed senescence, as compared to a control plant, said method comprising:
1) providing a recombinant nucleic acid encoding a polypeptide, wherein the polypeptide comprises a conserved domain having the amino acid sequence of SEQ ID NO: 7;
2) introducing into a plant cell the recombinant nucleic acid to produce a transgenic plant; and
3) growing the transgenic plant with an enhanced trait.

2. The method of claim 1, wherein the altered levels of at least one hexose sugar, the altered starch levels, the altered SPS activity and altered levels of at least one ureide is in leaves.

3. The method of claim 1, wherein the altered levels of at least one hexose sugar, the altered starch levels and the altered levels of at least one ureide is increased levels of at least one hexose sugar, increased levels of starch and increased levels of at least one ureide in R1 leaves, if the plant is a soybean plant, or in leaves from a plant at a developmental stage equivalent to R1 if the plant is not a soybean plant.

4. The method of claim 1, wherein the altered levels of at least one hexose sugar is increased levels of at least one hexose sugar in R5 pods, if the plant is a soybean plant, or in fruit from a plant at a developmental stage equivalent to R5 if the plant is not a soybean plant.

5. The method of claim 1, wherein the altered levels of at least one hexose sugar and the altered levels of at least one ureide is increased levels of at least one hexose sugar and increased levels of at least one ureide in R4 leaves or R5 leaves, if the plant is a soybean plant, or in leaves from a plant at a developmental stage equivalent to R4 or R5 if the plant is not a soybean plant.

6. The method of claim 1, wherein the altered levels of at least one hexose sugar is decreased levels of at least one hexose sugar in R6 leaves, if the plant is a soybean plant, or in leaves from a plant at a developmental stage equivalent to R6 if the plant is not a soybean plant.

7. The method of claim 1, wherein the altered starch levels is increased starch levels in R6 leaves, if the plant is a soybean plant, or in leaves from a plant at a developmental stage equivalent to R6 if the plant is not a soybean plant.

8. The method of claim 1, wherein the altered sucrose phosphate synthase activity is increased sucrose phosphate synthase activity in R4 leaves or R6 leaves, if the plant is a soybean plant, or in leaves from a plant at a developmental stage equivalent to R4 or R6 if the plant is not a soybean plant.

9. The method of claim 1, wherein the altered sucrose phosphate synthase activity is decreased sucrose phosphate synthase activity in R1 leaves, if the plant is a soybean plant, or in leaves from a plant at a developmental stage equivalent to R1 if the plant is not a soybean plant.

10. The method of claim 1, wherein the delayed senescence is delayed leaf senescence.

11. The method of claim 1, wherein the polypeptide further comprises a motif having an amino acid sequence selected from the group consisting of SEQ ID NO: 8, SEQ ID NO: 9, SEQ ID NO: 10, SEQ ID NO: 11 and SEQ ID NO:12.

12. The method of claim 1, wherein the polypeptide comprises an amino acid sequence selected from the group consisting of:
(a) SEQ ID NO: 4, SEQ ID NO: 5 or SEQ ID NO: 6; and
(b) a protein sequence at least 80% identical to any of SEQ ID NO: 4, SEQ ID NO: 5 or SEQ ID NO: 6.

13. The method of claim 1, wherein the recombinant nucleic acid comprises a nucleic acid sequence selected from the group consisting of:
(a) a nucleic acid sequence of SEQ ID NO: 1, SEQ ID NO: 2 or SEQ ID NO: 3;
(b) a nucleic acid sequence that hybridizes to SEQ ID NO: 1, SEQ ID NO: 2 or SEQ ID NO: 3 under conditions of 1×SSC, and 65° C.;
(c) a nucleic acid sequence at least 80% identical to any of SEQ ID NO: 1, SEQ ID NO: 2 or SEQ ID NO: 3; and
(d) a complement of any of (a)-(c).

14. The method of claim 1, wherein the method further comprises the step of:
4) selecting a transgenic plant by its ectopic expression of the polypeptide or its enhanced trait, as compared to the control plant.

15. A plant cell produced by the method of claim 1.

16. A plant produced from the plant cell of claim 15, wherein said plant is selected from the group consisting of soybean, cotton, canola, alfalfa, corn, rice, wheat, sunflower, barley, millet, sorghum, sugar beet, sugarcane and vegetables.

17. A seed produced from the plant of claim 16.

18. A commodity product from the plant of claim 16, selected from the group consisting of whole or processed seed, animal feed, oil, meal, mill, flour, flake, bran, protein concentrate, soy protein isolates, hydrolyzed soy protein, biomass and fuel products.


SEQUENCE LISTING (15 sequences)

SEQ ID NO: 1 - DNA, 678 bp, Arabidopsis thaliana
atggtgagcttttgcgagctttgtggtgccgaagctgatctccattgtgccgcggactctgccttcctctgccgttcttgtgacgctaagttccatgcctcaaattttctcttcgctcgtcatttccggcgtgtcatctgcccaaattgcaaatctcttactcaaaatttcgtttctggtcctcttcttccttggcctccacgaacaacatgttgttcagaatcgtcgtcttcttcttgctgctcgtctcttgactgtgtctcaagctccgagctatcgtcaacgacgcgtgacgtaaacagagcgcgagggagggaaaacagagtgaatgccaaggccgttgcggttacggtggcggatggcatttttgtaaattggtgtggtaagttaggactaaacagggatttaacaaacgctgtcgtttcatatgcgtctttggctttggctgtggagacgaggccaagagcgacgaagagagtgttcttagcggcggcgttttggttcggcgttaagaacacgacgacgtggcagaatttaaagaaagtagaagatgtgactggagtttcagctgggatgattcgagcggttgaaagcaaattggcgcgtgcaatgacgcagcagcttagacggtggcgcgtggattcggaggaaggatgggctgaaaacgacaacgtttag

SEQ ID NO: 2 - DNA, 726 bp, Glycine max
atgaagggtaagacttgcgagctttgtgatcaacaagcttctctctattgtccctccgattccgcatttctctgctccgactgcgacgccgccgtgcacgccgccaactttctcgtagctcgtcacctccgccgcctcctctgctccaaatgcaaccgtttcgccggatttcacatctcctccggcgctatatcccgccacctctcgtccacctgcagctcttgctccccggagaatccttccgctgactactccgattctctcccttcctcttctacctgcgtctccagttccgagtcttgctccacgaagcagattaaggtggagaagaagaggagttggtcgggttcctccgtgaccgacgacgcatctccggcggcgaagaagcggcagaggagtggaggatcggaggaggtgtttgagaaatggagcagagagatagggttagggttagggttaggggtaaacggaaatcgcgtggcgtcgaacgctctgagtgtgtgcctgggaaagtggaggtggcttccgttcagggtggctgctgcgacgtcgttttggttggggctgagattttgtggggacagagggctggcctcgtgtcagaatctggcgaggttggaggcaatatccggagtgccagttaagctgattctggccgcacatggcgacctggcacgtgtcttcacgcaccgccgcgaattgcaggaaggatggggcgagtcctag

SEQ ID NO: 3 - DNA, 732 bp, Glycine max
atgaagcccaagacttgcgagctttgtcatcaactagcttctctctattgtccctccgattccgcatttctctgcttccactgcgacgccgccgtccacgccgccaacttcctcgtagctcgccacctccgccgcctcctctgctccaaatgcaaccgtttcgccgcaattcacatctccggtgctatatcccgccacctctcctccacctgcacctcttgctccctggagattccttccgccgactccgattctctcccttcctcttctacctgcgtctccagttccgagtcttgctctacgaatcagattaaggcggagaagaagaggaggaggaggaggaggagtttctcgagttcctccgtgaccgacgacgcatctccggcggcgaagaagcggcggagaaatggcggatcggtggcggaggtgtttgagaaatggagcagagagatagggttagggttaggggtgaacggaaatcgcgtggcgtcgaacgctctgagtgtgtgcctcggaaagtggaggtcgcttccgttcagggtggctgctgcgacgtcgttttggttggggctgagattttgtggggacagaggcctcgccacgtgtcagaatctggcgaggttggaggcaatatctggagtgccagcaaagctgattctgggcgcacatgccaacctcgcacgtgtcttcacgcaccgccgcgaattgcaggaaggatggggcgagtcctag

SEQ ID NO: 4 - protein, 225 aa, Arabidopsis thaliana
MVSFCELCGAEADLHCAADSAFLCRSCDAKFHASNFLFARHFRRVICPNCKSLTQNFVSGPLLPWPPRTTCCSESSSSSCCSSLDCVSSSELSSTTRDVNRARGRENRVNAKAVAVTVADGIFVNWCGKLGLNRDLTNAVVSYASLALAVETRPRATKRVFLAAAFWFGVKNTTTWQNLKKVEDVTGVSAGMIRAVESKLARAMTQQLRRWRVDSEEGWAENDNV

SEQ ID NO: 5 - protein, 241 aa, Glycine max
MKGKTCELCDQQASLYCPSDSAFLCSDCDAAVHAANFLVARHLRRLLCSKCNRFAGFHISSGAISRHLSSTCSSCSPENPSADYSDSLPSSSTCVSSSESCSTKQIKVEKKRSWSGSSVTDDASPAAKKRQRSGGSEEVFEKWSREIGLGLGLGVNGNRVASNALSVCLGKWRWLPFRVAAATSFWLGLRFCGDRGLASCQNLARLEAISGVPVKLILAAHGDLARVFTHRRELQEGWGES

SEQ ID NO: 6 - protein, 243 aa, Glycine max
MKPKTCELCHQLASLYCPSDSAFLCFHCDAAVHAANFLVARHLRRLLCSKCNRFAAIHISGAISRHLSSTCTSCSLEIPSADSDSLPSSSTCVSSSESCSTNQIKAEKKRRRRRRSFSSSSVTDDASPAAKKRRRNGGSVAEVFEKWSREIGLGLGVNGNRVASNALSVCLGKWRSLPFRVAAATSFWLGLRFCGDRGLATCQNLARLEAISGVPAKLILGAHANLARVFTHRRELQEGWGES

SEQ ID NO: 7 - protein, 47 aa, Artificial Sequence
  Synthetic polypeptide
XXXXXCELCXXXAXLXCXXDSAFLCXXCDAXXHAXNFLXARHXRRXX

SEQ ID NO: 8 - protein, 6 aa, Artificial Sequence
  Synthetic polypeptide
TCXSXS

SEQ ID NO: 9 - protein, 9 aa, Artificial Sequence
  Synthetic polypeptide
SSSXXCXSS

SEQ ID NO: 10 - protein, 9 aa, Artificial Sequence
  Synthetic polypeptide
RVXXAXXFW

SEQ ID NO: 11 - protein, 12 aa, Artificial Sequence
  Synthetic polypeptide
QNLXXXEXXXGV

SEQ ID NO: 12 - protein, 5 aa, Artificial Sequence
  Synthetic polypeptide
EGWXE

SEQ ID NO: 13 - protein, 46 aa, Arabidopsis thaliana
MVSFCELCGAEADLHCAADSAFLCRSCDAKFHASNFLFARHFRRVI

SEQ ID NO: 14 - protein, 47 aa, Glycine max
MKGKTCELCDQQASLYCPSDSAFLCSDCDAAVHAANFLVARHLRRLL

SEQ ID NO: 15 - protein, 47 aa, Glycine max
MKPKTCELCHQLASLYCPSDSAFLCFHCDAAVHAANFLVARHLRRLL